(19) United States
(12) Patent Application Publication
(10) Pub. No.: US 2012/0197206 A1
(43) Pub. Date: Aug. 2, 2012

(54) ADHESIVE DRESSING WITH RESEALABLE COVER

(76) Inventor: Bradley J. Glenn, Danville, CA (US)

(21) Appl. No.: 13/360,232

(22) Filed: Jan. 27, 2012

Related U.S. Application Data
(60) Provisional application No. 61/457,206, filed on Jan. 31, 2011.

(51) Int. Cl. A61M 25/02

(52) U.S. Cl. 604/180; 604/174

(57) ABSTRACT

A dressing assembly is provided including a cover overlying an inner cavity, spacing portions of the cover away from skin adjacent where the cover is coupled to the skin The cover includes a side wall extending up from a footing surface adapted to be placed adjacent the skin The side wall extends up to a top wall overlying the cavity. A door is provided in the cover through which the inner cavity can be accessed. The door can be sealingly closed and opened to selectively provide access to the cavity or to be closed and maintain a sanitary environment within the cavity. An adhesive membrane is provided adjacent the skin and beneath the footing surface with the adhesive membrane defining a floor of the cavity. A transcutaneous medical device can have portions thereof housed within the cavity where the cover protects it and keeps it sanitary.

### CROSS-REFERENCE TO RELATED APPLICATIONS

This application claims benefit under Title 35, United States Code §119(e) of U.S. Provisional Application No. 61/457,206 filed on Jan. 31, 2011.

### FIELD OF THE INVENTION

The following invention relates to dressings for placement on the skin of a patient such as to maintain a sterile environment at a particular location, such as an incision point or other access point through the skin More particularly, this invention relates to dressing assemblies for covering and protecting an access point which has a subcutaneous or transcutaneous vascular access device located there, with the cover configured to protect the proximal portions of the transcutaneous vascular access device in a sterile compartment which can be selectively accessed.

### BACKGROUND OF THE INVENTION

This invention relates to an improved method of dressing a transcutaneous catheter such as a peripherally inserted central catheter (PICC), central line, peripheral IV or implanted port. Although these venous access devices are used for examples in this description, the same concepts can be applied to any transcutaneous catheter such as a catheter used to access other body lumens.

Current methods to dress a PICC generally include separate devices to accomplish three separate functions: sterile covering of the access point at the skin, securement of the catheter to prevent dislodgment, and containment of the free end of the catheter to prevent tugging or pulling of the catheter end.

One product often used to sterilely cover a PICC access point in the skin is a TEGADERM dressing provided by the 3M Company of St. Paul, Minn. Such dressings are in the form of a semi-permeable film of a material such as polyurethane which can be placed upon the skin overlying the PICC body to protect it and keep it sanitary.

One product for holding the PICC line in place is provided by C. R. Bard, Inc. of Murray Hill, N.J. under the trademark STATLOCK. This device adheres to the skin and has structures therein designed to capture and hold the wings of the PICC body thereon. While helpful, the PICC body is still largely exposed and susceptible to contamination or impact with adjacent objects.

Current methods to contain the free external ends of the PICC include old fashioned tape and gauze as well as various sorts of ad hoc tubular sock-like fabric sleeves which are slipped over the patients arm to hold the external portions of the PICC against the skin Such equipment doe not effectively store the external ends of a PICC in a sanitary fashion.

Because of the multitude of devices that are currently used to dress a PICC, the procedure for dressing change is quite cumbersome and time consuming for the health care provider. In addition, the combination of devices used is unsightly for the patient and is not conducive to performing normal activities of daily living. Clearly, a need exists for a method to provide these three functions in a more simple and effective manner

### SUMMARY OF THE INVENTION

With this invention, a dressing assembly is provided to protect an access site where tubing or a needle is passing transcutaneously into the skin The dressing assembly, in a preferred form preferably includes as a foundational layer an adhesive membrane which adheres to the skin at the access point. A cover attaches to this adhesive membrane (or directly to the skin if no adhesive membrane is provided). The cover has side walls which extend up from the adhesive membrane and come together at a top wall overlying an inner cavity which is isolated between the top wall of the cover and the adhesive membrane (or the skin itself). A door is provided on a portion of the cover spaced from the skin This door is openable and closable in a resealable fashion. Thus, tubing and other equipment within the inner cavity can be accessed through the door and the door can then be resealed closed to maintain a sterile environment when access is not required.

Preferably, the cover has a somewhat dome-like shape with the side walls tapering upwardly and inwardly at least somewhat. With such a shape, the cover is less likely to be snagged by clothing or other objects when inadvertently impacted adjacent the incision site. The cover is also beneficially made from a flexible material, such as a solid foam material to allow the cover to flex when impacted somewhat, and to also allow the cover to distort in shape somewhat along with bending of the skin

The cover can be provided in a variety of different configurations such as the configurations depicted in the preferred embodiment and alternative embodiments disclosed herein, as well as other shapes and sizes to accommodate various different incision sites and an amount of catheter tubing or other equipment to be stored within an inner cavity thereof.

The adhesive membrane can be in the form of hydrocolloid material, a transparent semi-permeable membrane, polyurethane film, or some combination thereof. The adhesive membrane could alternatively be made of other thin flexible material that can adhesively hold to the skin The adhesive membrane preferably has an aperture therein, inside the inner cavity, through which PICC line tubing or other transcutaneous medical device portions may pass.

### OBJECTS OF THE INVENTION

Accordingly, a primary object of the present invention is to provide a skin dressing which has an inner cavity defining a sterile environment in which transcutaneous access tubing or other equipment can be stored over a transcutaneous access point.

Another object of the present invention is to provide a dressing for protecting a transcutaneous access point in the skin from inadvertent mis-positioning of external portions of the transcutaneous vascular access system.

Another object of the present invention is to provide a dressing assembly which can protect external portions of a vascular access device while flexibly bending along with skin of the patient to maximize comfort and maintenance of contact with the skin when flexing.

Another object of the present invention is to provide a method for maintaining a secure and sterile environment adjacent an access point for a transcutaneous vascular access device.

Another object of the present invention is to provide a desirable dressing having an attractive and minimally noticeable appearance for overlying a transcutaneous vascular access device.

Another object of the present invention is to provide a dressing to decrease the discomfort experienced by a patient when a vascular access device has been implanted beneath or through an access point in the skin

Other further objects of the present invention will become apparent from a careful reading of the included drawing figures, the claims and detailed description of the invention.

### BRIEF DESCRIPTION OF THE DRAWINGS

FIG. 1 is a perspective view of a dressing assembly according to a preferred embodiment of this invention, in position upon a patient's skin at an access site where a transcutaneous vascular access device might be located extending through the skin

FIG. 2 is a perspective view similar to FIG. 1, but with a door of a cover of the dressing assembly open to reveal portions of an inner cavity and portions of a transcutaneous vascular access device, including catheter tubing stored within the inner cavity in a protected and sterile environment.

FIG. 3 is a full sectional view of that which is shown in FIG. 2, taken along lines 3-3 of FIG. 2.

FIG. 4 is a full sectional view of an alternative embodiment of that which is shown in FIG. 3, where a subcutaneous vascular access port is protected along with a needle shown accessing the subcutaneous vascular access port, rather than a transcutaneous catheter.

FIG. 5 is a top plan view of an alternative geometry for the dressing assembly that is cylindrical according to an alternative embodiment of this invention.

FIG. 6 is a side elevation view of that which is shown in FIG. 5.

FIG. 7 is a side elevation view of a further alternative frusto-conical dressing assembly according to an alternative embodiment of this invention.

FIG. 8 is a side elevation view similar to that which is shown in FIG. 7, but with the dressing assembly having a domed shape in a further alternative embodiment of this invention.

FIG. 9 is a top plan view of this invention according to a still further alternative embodiment having a frusto-conical shape and with an inner cavity having a unique shape and a uniquely shaped door for accessing the inner cavity.

FIG. 10 is a side elevation view of that which is shown in FIG. 9.

FIG. 11 is a top plan view of the dressing assembly of this invention according to a preferred embodiment and showing the door and seal layer of the door in a closed position.

FIG. 12 is a bottom plan view of that which is shown in FIG. 9 and including the adhesive membrane and release liners covering the adhesive membrane before its use.

FIG. 13 is a top plan view of the cover portion only of that which is shown in FIG. 11.

FIG. 14 is a bottom plan view of that which is shown in FIG. 13.

FIG. 15 is a perspective view of that which is shown in FIG. 13.

FIG. 16 is a bottom plan view of the seal layer of the door.

FIG. 17 is a side elevation view of that which is shown in FIG. 16.

FIG. 18 is a perspective view of that which is shown in FIG. 16.

FIG. 19 is a top plan view of the adhesive membrane of the dressing assembly shown alone, and featuring an aperture therein for passage of catheter tubing or other medical device portions therethrough and into the inner cavity of the cover.

FIG. 20 is a bottom plan view of that which is shown in FIG. 19, and with one of the release liners located upon the adhesive supporting undersurface of the adhesive membrane.

FIG. 21 is a perspective view of that which is shown in FIGS. 19 and 20, but from a viewpoint beneath the dressing assembly to further reveal the configuration of the adhesive membrane and release liners on the adhesive membrane before it is placed on the skin of a patient, and with cover portions of the dressing assembly shown in broken lines.

### DESCRIPTION OF THE PREFERRED EMBODIMENT

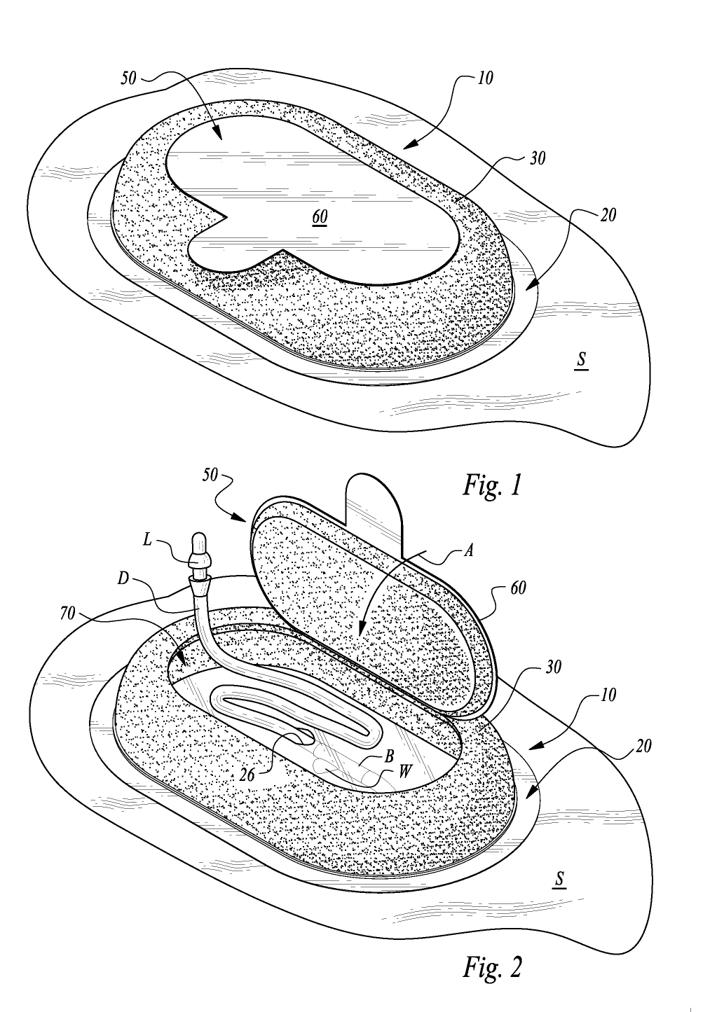

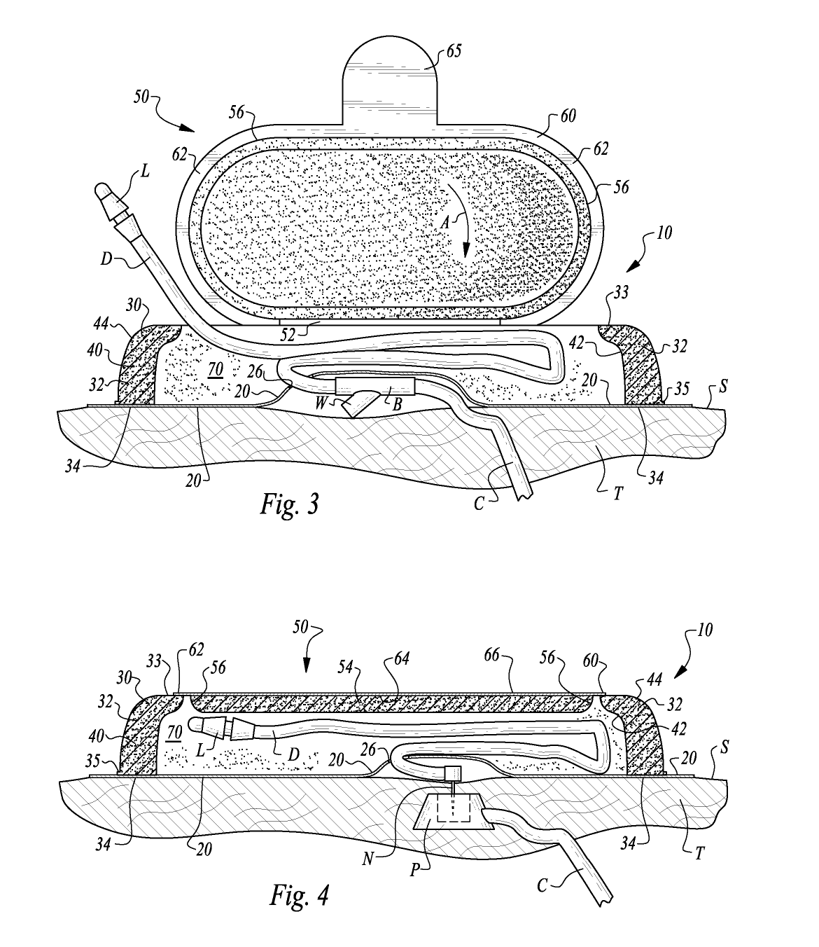

Referring to the drawings, wherein like reference numerals represent like parts throughout the various drawing figures, reference numeral 10 is directed to a dressing assembly (FIGS. 1-4, 11 and 12) which can be attached to skin S of a patient, adjacent a transcutaneous access point, such as where a PICC line catheter C passes (FIG. 3) through the skin S or where a port P (FIG. 4) is accessed through the skin S or where some other transcutaneous or subcutaneous medical device is located. The dressing assembly 10 protects this access point while also maintaining a secure and sanitary cavity 70 where tubing D or other equipment can also be stored.

In essence, and with particular reference to FIGS. 1-4, basic details of the dressing assembly 10 are described, according to a preferred embodiment. The dressing assembly 10 preferably has two main components including an adhesive membrane 20 portion and a cover 30 portion. The adhesive membrane 20 is optional, but preferably is provided to provide a sanitary layer upon the skin S upon which the cover 30 is mounted. An aperture 26 through the adhesive membrane 20 provides a point through which a catheter C or tubing D can pass through the adhesive membrane 20 and into the inner cavity 70 above the adhesive membrane 20 and beneath the cover 30.

The cover 30 is attached to the adhesive membrane 20 around a perimeter thereof (or directly attached to the skin S) and extends up away from the skin S to provide the inner cavity 70 beneath the cover 30. The cover 30 is preferably formed of foam material 40 or other flexible material so that it can flex with the skin S, and so that the cover 30 can resiliently absorb impacts associated with clothing or bumping into adjacent objects when a patient is wearing the dressing assembly 10 on the patient's skin S. A door 50 is provided in the cover 30. The door 50 can be selectively opened and closed to provide selective access into the inner cavity 70. The door 50 preferably includes a seal layer 60 which has an adhesive thereon which allows the door 50 to seal to the cover 30 in a temporary fashion when closed. The inner cavity 70 is thus maintained in a sanitary manner

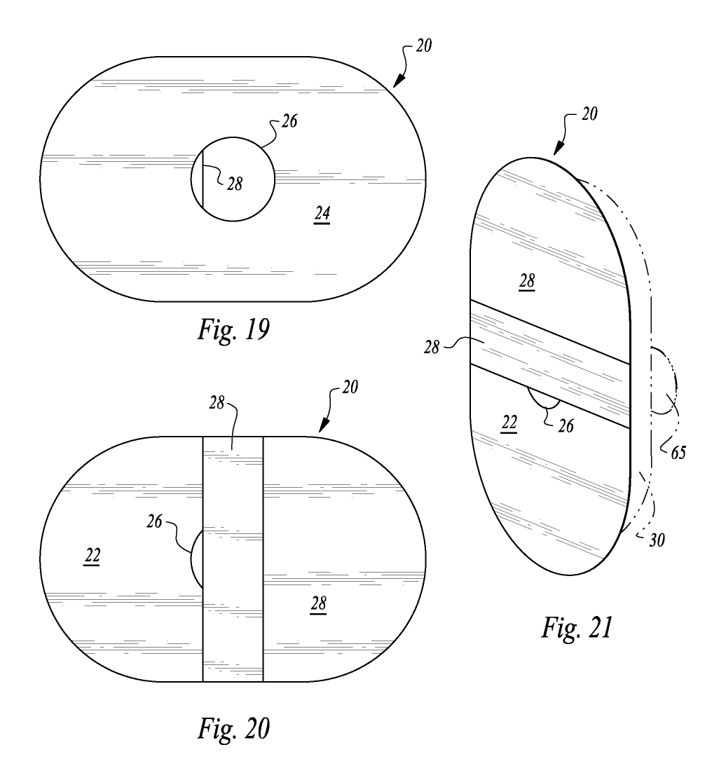

With particular reference to FIGS. 3, 4 and 19-21, details of the adhesive membrane 20 of the dressing assembly 10 are described, according to a most preferred embodiment. The adhesive membrane 20 is not required for utilization of the cover 30 of this invention. Rather, the cover 30 can be directly attached to the skin S. However, most preferably the cover 30 is affixed to an over surface 24 of the adhesive membrane 20 opposite an undersurface 22. The undersurface 22 is provided with an adhesive thereon which allows the adhesive membrane 20 to attach securely to the skin S of the patient.

The adhesive membrane 20 itself can be formed of a variety of different materials to provide the basic function according to this invention. In one form, the adhesive membrane can be provided as hydrocolloid material. As another option, the adhesive membrane 20 can be provided as a transparent (or semi or non-transparent) semi-permeable membrane. In another embodiment, the adhesive membrane can be provided as polyurethane film. Preferably, the adhesive membrane 20 is attached to the cover 30 in advance of use of the entire dressing assembly 10, in a most preferred form of this invention. Release liners 28 are placed over the undersurface 22 of the adhesive membrane 20 to protect the adhesive until it is ready to be bonded to the skin S.

Preferably, an aperture 26 is provided in the adhesive membrane 20. This aperture 26 preferably has a size smaller than an overall size of the floor of the inner cavity 70. The aperture 26 is located defining a portion of the floor of the inner cavity 70 inboard of walls of the cover 30. In one embodiment, the aperture 26 is generally circular. The aperture 26 could as an option not be provided, and a transcutaneous vascular access device can merely be passed through the adhesive membrane 20 by piercing the adhesive membrane 20, along with utilization of the transcutaneous vascular access device. For instance, a needle N (FIG. 4) accessing a port P in tissue T beneath the skin S could merely pierce the adhesive membrane 20 when the needle N is accessing the port P. Most preferably, the aperture 26 is provided and tubing D or a catheter C is routed through the aperture 26 before passing through the skin S or being coupled to a structure (e.g. a needle) which is passing through the skin S.

When the transcutaneous vascular access device is in the form of a PICC line, a PICC body B is preferably provided adjacent the skin S and beneath the adhesive membrane 20. In this way, the PICC body B and associated wings W are held in place against the skin S by the adhesive membrane 20, holding an orientation of the body B and wings W fixed relative to the skin S. Tubing D coupled to the PICC body B then passes through the aperture 26 and into the inner cavity 70. A luer cap L or other closure would typically be provided on an end of such tubing D. When a medical professional needs to access the catheter C or other tubing D, the door 50 is opened and the medical professional can access the inner cavity 70 where such tubing D is stored. Once such access is no longer required, tubing D can be tucked back into the inner cavity 70 and the door 50 re-closed (along arrow A of FIGS. 2 and 3) to reseal the inner cavity 70 and to maintain a sanitary environment therein.

The adhesive membrane 20 preferably has a size larger than a perimeter of the cover 30. This amount of extra size for the adhesive membrane 20 can be a uniform amount beyond a perimeter of the cover 30, or some other non-uniform amount. For instance, the adhesive membrane 20 can be a larger rectangular strip of material while the cover 30 has an oval shape. In the embodiments shown (FIGS. 1-4) a uniform amount of adhesive membrane 20 extends beyond a perimeter of the cover 30.

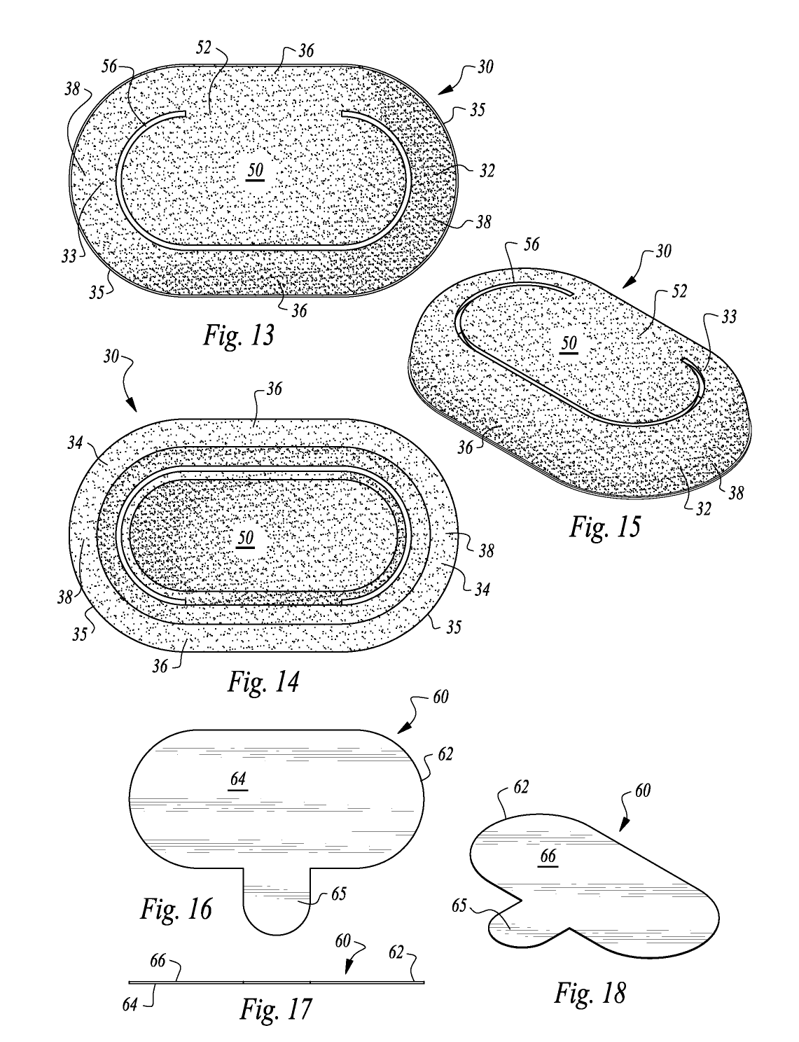

With particular reference to FIGS. 3, 4 and 13-15, details of the cover 30 are described, according to a most preferred embodiment. The cover 30 is preferably a unitary mass of material which acts to overlie the access point where the subcutaneous or transcutaneous vascular access device is located. The cover 30 is configured to have side walls 32 which extend up from a footing surface 34 to a top wall 33. The footing surface 34 is that surface configured to be affixed to the over surface 24 of the adhesive membrane 20. As an alternative, the footing surface 34 can itself have a skin S compatible adhesive thereon so that the footing surface 34 can allow the cover 30 to be directly attached to the skin S without the adhesive membrane.

The side walls 32 define an outer perimeter 35 of the cover 30 adjacent where the side walls 32 terminate at the footing surface 34. This outer perimeter 35 defines a surface area covered by the cover 30. The side walls 32 preferably taper inwardly somewhat. These side walls 32 in a preferred form of the invention include two long sides 36 and two curving sides 38 so that the overall cover 30 has an oval shape (FIGS. 1, 2, 11 and 12.

The top wall 33 is preferably substantially planar and located on a side of the side walls 32 opposite the footing surface 34. The top wall 33 has an opening therein which is selectively closed by the door 50. This opening in the top wall 33 defines a jamb for the door 30 against which the door 50 is located when the door 50 is closed.

The cover 30 preferably has a thickness which is somewhat variable. For instance, the cover 30 can be thickest at the footing surface 34 and tapered to be slightly thinner as it extends up towards the top wall 33.

The cover 30 is preferably formed of a foam material 40 which exhibits resiliency and flexibility. The foam material 40 is bounded by an inner surface 42 facing the inner cavity 70 and an outer surface 44 opposite the inner surface 42. The foam material 40 can be any of a variety of foam materials, but most preferably exhibits biocompatibility, the ability to resist holding of contaminates or bacteria thereon, be easily cleaned and exhibit the resilient characteristics desired for the cover 30.

The foam material 40 preferably is sufficiently resilient that it holds its shape under gravity loads. However, when the cover 30 is bumped, the flexibility of the foam material 40 allows the cover 30 to collapse somewhat under such impact loads. When the impact loads subside, the foam material 40 is sufficiently resilient to cause the cover 30 to resiliently return to an original shape. Also, the foam material 40 is preferably sufficiently flexible that when the cover 30 is caused to bend, such as when flexing the skin S to which the dressing assembly 10 is attached (e.g. at an inner side of the elbow's arm where the dressing assembly 10 might often be located), the cover 30 can resiliently bend somewhat and return to its original shape after such bending. The foam material 40 (FIGS. 3 and 4) is preferably homogenous, but could be layered, formed with cellular voids, or otherwise exhibit irregular density.

With continuing reference to FIGS. 1-4 and 13-18, details of the door 50 are described, according to this most preferred embodiment. The door 50 provides a preferred form of means to selectively access the inner cavity 70 of the dressing assembly 10. This door 50 is preferably located in the top wall 33 of the cover 30, but could be in the side walls 32 or provided by lifting a portion of the cover 30 off of the adhesive membrane 20 at the footing surface 34. Other forms of selectively closable entrances other than the door 50 could also be used, including making the entire cover 30 at least partially removable from the adhesive membrane 20 (or the skin S) to access the inner cavity. The cover can be in the form of a flap of foam or other material which could lay flat against the adhesive membrane 20 (or the skin S) or have a domed form spacing portions of the cover away from the adhesive membrane 20 (or skin S). When the cover is in the form of a flap, an opening can optionally be defined by a gap between such a flap and the adhesive membrane 20 or between the flap and a second layer of foam or other material with the inner cavity at least partially located therebetween. Such a gap could be held closed by adhesive or other fasteners such as those disclosed herein.

The door 50 has a shape preferably filling the opening in the top wall 33. This door 50, in a preferred embodiment, is formed with a core 54 of the same foam material 40 forming the cover 30, with this core 54 coupled to the top wall 33 through a hinge 52. An outer edge 56 of the door 50 is located adjacent the top wall 33 so that the door 50 can close the cover 30 when the door 50 is in a closed position. The door 50 can pivot about the hinge 52 (arrow A of FIGS. 2 and 3) to selectively open and close the cover 30 for accessing or sealing off the inner cavity 70.

The door 50 preferably includes a seal layer 60 defining an upper portion of the door 50. This seal layer 60 can be in the form of an adhesive layer of material. The seal layer 60 in such an embodiment is a thin flexible sheet of material having a perimeter 62 similar to a perimeter of the core 54 of the door 50, but slightly greater in size. A lower surface 64 of the seal layer 60 includes an adhesive thereon. Portions of this adhesive allow a lower surface 64 to be bonded to the core 54 of the door 50. Portions of the lower surface 64 adjacent the perimeter 62 overlie the top wall 33 of the cover 30 and allow this portion of the lower surface 64 adjacent the perimeter 62 to seal the door 50 closed on the cover 30. If desired, the adhesive can vary so that the seal layer 60 is held permanently to the core 54 of the door 50 but releasably and re-sealably attach to the top wall 33 of the cover 30 at the perimeter 62.

An upper surface 66 is provided opposite the lower surface 64 and defines a side of the layer 60 most distant from the inner cavity 70. A tab 65 preferably extends from one side of this seal layer 60. Preferably, this tab 65 does not have adhesive thereon. The tab 65 extends away from the cover 30 somewhat and provides a convenient structure which can be grasped by fingers of a user to peel the seal layer 60 of the door 50 off of the top wall 33 of the cover 30 to open the door 50, when desired. The tab 65 can also be used to close the door 50 when desired. While the tab 65 is shown as part of the seal layer 60 it could instead (or in addition) be formed by a portion of the core 54 of the door 50.

As an alternative, the seal layer 60 can be integrally formed with the door 50. As another alternative, the door 50 can include structures other than the seal layer 60 to allow the door 50 to be secured in a closed position adjacent the top wall 33 of the cover 30. A variety of different fasteners could be utilized for this purpose including hook and loop fasteners (such as those provided under the trademark VELCRO by Velcro Industries B.V. of Curacao, Netherlands) or snaps, buttons, laces, zippers or other fasteners known to exist in the prior art or developed in the future.

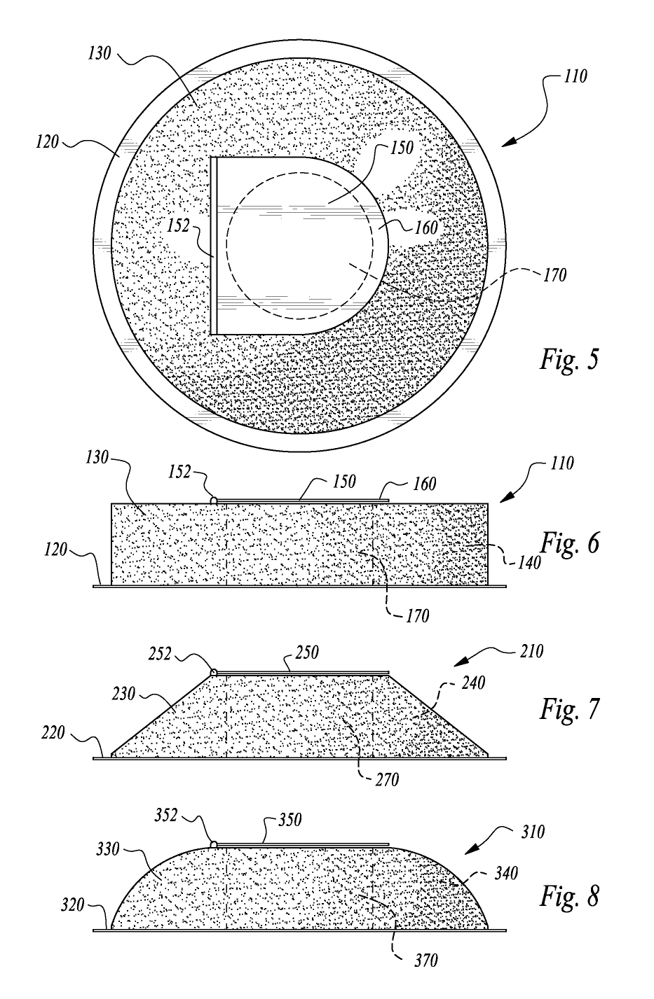

With particular reference to FIGS. 5-10, various alternative embodiment assemblies 110, 210, 310, 410 are described as variations on the preferred embodiment described above. In FIGS. 5 and 6 a cylindrical dressing assembly 110 is depicted. A circular adhesive membrane 120 defines a lower skin S contacting portion of this cylindrical assembly 110. A cover 130 is coupled to the adhesive membrane 120 (or configured to be directly attachable to the skin S). A door 150 is provided on a portion of the cover 130 opposite the adhesive membrane 120. This door 150 is curved on one end and straight on a second end where a hinge 152 is provided. A seal layer 160 causes the door 150 to be able to sealingly attach to portions of the cover 130 adjacent the door 150. A central cavity 170 is located beneath the door 150 and between the cover 130 and the adhesive membrane 120. The central cavity 170 is depicted by broken lines in FIGS. 5 and 6.

With particular reference to FIG. 7, details of a frusto-conical dressing assembly 210 are described. In this embodiment, an adhesive membrane 220 of generally circular form is provided with a frusto-conical shaped cover 230 coupled thereto on a side of the adhesive membrane 220 opposite the skin S. This cover 230 has sides which taper up from the adhesive membrane 220. An upper portion of the cover 230 is essentially flat. A door 250 is provided mounted by a hinge 252 to upper portions of the cover 230. A central cavity 270 is provided within the cover 230, beneath the door 250 and above the adhesive membrane 220.

With particular reference to FIG. 8, details of a domed dressing assembly 310 are described. This domed dressing assembly 310 includes an adhesive membrane 320 which is generally circular in form and with a cover 330 coupled to the adhesive membrane 320. This cover 330 has a domed shape which exhibits a continuous curve from the adhesive membrane 320 up to an upper portion of the cover 330. A door 350 is provided on an upper portion of the cover 330, which is attached by a hinge 352 to the cover 330 to provide selective access to a central cavity 370 within the domed dressing assembly 310.

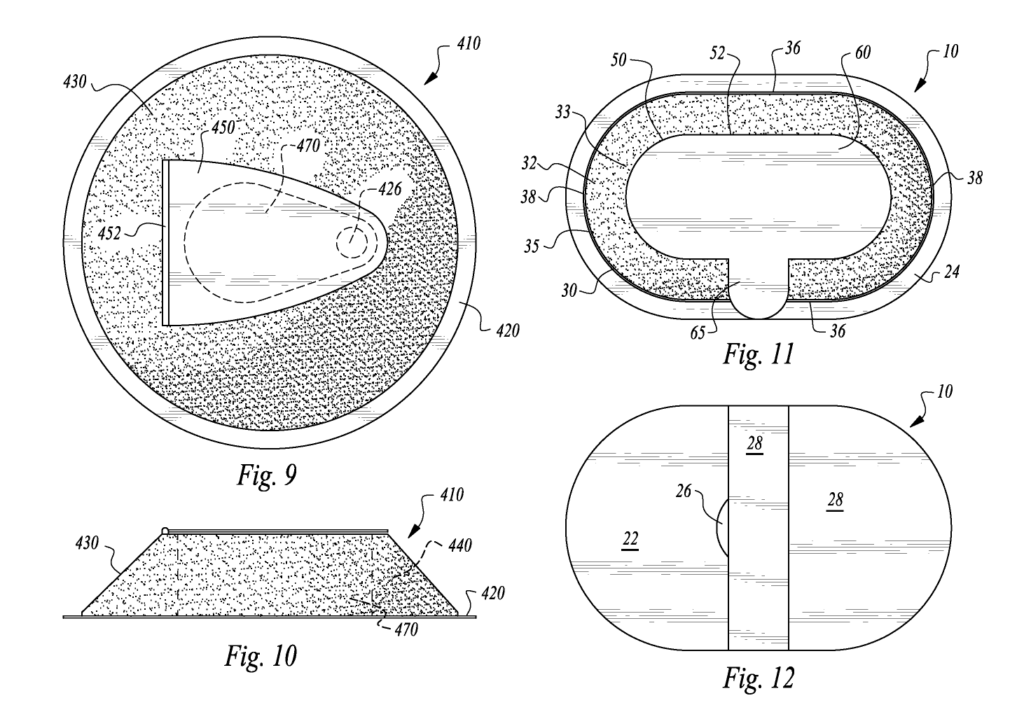

With particular reference to FIGS. 9 and 10, details of an ovoid dressing assembly 410 featuring an inner cavity 470 which is somewhat oval in shape, is depicted. A circular adhesive membrane 420 is provided with an aperture 426 in a portion of this adhesive membrane 420. A cover 430 extends up from the adhesive membrane 420 preferably having a generally frusto-conical shape (FIG. 10) extending up to a generally circular and flat upper portion. Foam 440 defines the cover 430 providing the dressing assembly 410 with a resilient character. The door 450 is attached by way of a hinge 452 to upper portions of the cover 430 to provide selective access to an inner cavity 470.

The inner cavity 470 in this embodiment has curving ends with one end having a greater diameter than the other end. The aperture 426 in this embodiment is off-center from a center of the inner cavity 470 and a center of the cover 430. Such an arrangement can be advantageous where it is desirable that the access point through the skin S be off-center from a location where a structure such as a PICC body B is to be located (FIG. 3) so that the PICC body B can be centrally located within the inner cavity 470 without requiring the catheter C to transition too abruptly from extending substantially horizontal to a surface of the skin S to extending somewhat perpendicular to the skin S to pass through the skin S.

With particular reference to FIGS. 1-4, 11 and 12, details of the use and operation of the dressing assembly 10 of this invention are described in one particular method of use thereof. Initially, a patient will typically undergo a medical procedure to have a subcutaneous or transcutaneous vascular access device implanted at an appropriate location on the body of the patient. This vascular access device can be in the form of a port P (FIG. 4) which is located subcutaneously within tissue T beneath the skin S. A catheter C extends from the port P to a body lumen, such as a vein. In a simplest form of this invention, the dressing assembly 10 can merely be placed over a location where this port P is located even though the port P is entirely subcutaneous. The skin S is thus protected by the dressing assembly 10 to keep an access point on the skin S clean and protected. The dressing assembly 10 can include the adhesive membrane 20 and cover 30 such as those depicted in various embodiments above, or could conceivably merely be provided in the form of a cover 30 without the adhesive membrane 20.

Where the vascular access device is a transcutaneous vascular access device, reference is made to the depiction of a PICC body B (FIG. 3) located above the skin S and adjacent the skin S, with a catheter C extending from the PICC body B through an access point in the skin S to tissue T beneath the skin S, and on to a vein or other body lumen. The PICC body B typically includes wings W thereon. According to a preferred method of the invention, the dressing assembly 10 includes both the adhesive membrane 20 and the cover 30. The adhesive membrane 20 is located above the PICC body B to help secure the PICC body B and wings W between the adhesive membrane 20 and the skin S, such as to keep the PICC body B and wings W from moving relative to the skin S. Tubing D above the PICC body B can be stored within the inner cavity 70 of the dressing assembly 10 with the tubing D closed off by a luer cap L or other closure.

Initially, after the vascular access device has been implanted, the dressing assembly 10 is prepared by removing release liners 28 from the undersurface 22 of the adhesive membrane 20 (FIG. 12). Typically, the door 50 is then opened (FIGS. 2 and 3) and the dressing assembly 10 has the tubing D of the vascular access device threaded through the aperture 26 in the adhesive membrane 20 and up through the door 50. Simultaneously, the dressing assembly 10 has the undersurface 22 of the adhesive membrane 20 placed down against the skin S adjacent the access point in the skin S

If the vascular access device is in the form of a PICC line, the PICC body B and wings W are typically trapped between the adhesive membrane 20 and the skin S (FIG. 3). If the vascular access device is in the for of a port P (FIG. 4), tubing D can be threaded through the aperture 26 while the needle N remains beneath the adhesive membrane 20. As an alternative, the dressing assembly 10 can be placed over the access point above the port P when the port P is not currently being accessed by a needle N. Finally, any tubing D is gathered together and tucked into the inner cavity 70 and the door 50 is closed (along arrow A of FIGS. 2 and 3). The tubing D and any proximal portions of the transcutaneous vascular access device above the skin S are thus kept secure and sanitary within this inner cavity 70.

Because the cover 30 of the dressing assembly 10 is formed of resilient material such as the foam material 40, the dressing assembly 10 can flex with the skin S of the patient and also can resiliently absorb impacts associated with coming into contact with clothing or bumping into other objects. The dressing assembly 10 thus protects the vascular access device from becoming dislodged or otherwise interrupted in optimal performance and position through protection provided by the dressing assembly 10. When a medical professional is required to gain access to the vascular system or other body lumen of the patient, the medical professional would again open the door 50 by grabbing the tab 65 and pivoting the door 50 about the hinge 52. The tubing D can then be accessed as called for by the medical procedure to be performed.

After that procedure is performed, the tubing D can again be tucked into the inner cavity 70 and the door 50 closed and resealed. When the vascular access device is a port P, optionally no tubing D would be required to be contained within the inner cavity 70 and stored therein. Rather, a dressing assembly 10 would merely provide protection for the subcutaneous port P and skin S where the access point is located. A sanitary environment and protection, as well as concealment, is thus provided.

When the port P is to be accessed, the door 50 is opened and then a needle N is utilized to pass through the skin S and into the port P. The needle N could pass through the aperture 26 and the adhesive membrane 20 or merely puncture the adhesive membrane 20 adjacent the skin S before passing into the port P.

This disclosure is provided to reveal a preferred embodiment of the invention and a best mode for practicing the invention. Having thus described the invention in this way, it should be apparent that various different modifications can be made to the preferred embodiment without departing from the scope and spirit of this invention disclosure. When structures are identified as a means to perform a function, the identification is intended to include all structures which can perform the function specified. When structures of this invention are identified as being coupled together, such language should be interpreted broadly to include the structures being coupled directly together or coupled together through intervening structures. Such coupling could be permanent or temporary and either in a rigid fashion or in a fashion which allows pivoting, sliding or other relative motion while still providing some form of attachment, unless specifically restricted.

What is claimed is:

1. A dressing for covering an access point where a transcutaneous medical device passes through the skin, the dressing comprising in combination:
a cover, said cover having an outer perimeter with a footing surface adjacent the outer perimeter;
said footing surface adapted to be coupled to skin adjacent the access point;
the footing surface surrounding a majority of the access point;
a side wall extending from said footing surface in a direction at least partially away from the skin when the footing surface is adjacent the skin;
a top wall extending from said side wall of said cover;
an inner cavity inboard of said side wall of said cover and on a side of said top wall adjacent the skin when said footing surface is located adjacent the skin, said inner cavity sized to house at least a portion of a transcutaneous medical device outside of the access point; and
a selectively closable entrance into said inner cavity for access to said portion of said transcutaneous medical device outside of the patient.

2. The apparatus of claim 1 wherein said cover is at least partially formed of a flexible material.

3. The apparatus of claim 1 wherein said entrance includes a door on said cover, said inner cavity accessible through said door when said door is open.

4. The apparatus of claim 3 wherein said door has a perimeter with adhesive thereon, said perimeter of said door sized larger than an opening in said cover adjacent said door, with said adhesive perimeter of said door adapted to abut said cover adjacent said door to sealingly close said door, said perimeter of said door formed with a resealable adhesive which allows said door to be repeatedly opened and sealingly closed.

5. The apparatus of claim 1 wherein an adhesive membrane is coupled to said footing surface and spanning at least a portion of a floor of said inner cavity spaced from said top wall, said adhesive membrane having an adhesive undersurface adapted to contact and adhere to skin

6. The apparatus of claim 5 wherein said adhesive membrane includes an aperture therein, said aperture smaller than a size of said floor of said inner cavity, said aperture in said adhesive membrane defining a portion of said floor of said inner cavity.

7. The apparatus of claim 1 wherein said top wall is spaced from the skin when said footing surface is adjacent the skin

8. A method for accessing a body lumen of a patient transcutaneously, the method including the steps of:
providing an elongate tube having a distal end and a proximal end opposite the distal end;
forming at least one incision at an access point in skin of a patient;
routing the tube through the at least one incision at the access point and into a body lumen with the distal end within the body lumen and the proximal end outside of the access point;
placing a dressing assembly over the access point on the skin of the patient, the dressing assembly including a cover substantially enclosing an inner cavity; and
locating the proximal end of the elongate tube within the inner cavity.

9. The method of claim 8 including the further step of allowing the cover to flex when skin adjacent the cover flexes, with the cover secured to skin surrounding the access point.

10. The method of claim 9 including the further step of locating the access point near an inner elbow of the patient and allowing the cover to flex with the flexing of the arm of the patient.

11. The method of claim 8 including the further step of providing an adhesive membrane between the cover and the skin, the adhesive membrane covering a majority of a floor of the inner chamber of the cover, with the elongate tube passing through the adhesive membrane.

12. The method of claim 11 including the further step of locating an aperture within the adhesive membrane and at the access point, said aperture having a size smaller than a size of the floor of the inner cavity; and
routing the elongate tube through the aperture.

13. The method of claim 8 including the further step of configuring the cover to include a resealable door on a portion of said cover spaced from the skin of the patient, the door adapted to transition between an open configuration when the inner cavity can be accessed and a closed configuration where the inner cavity is isolated from a surrounding environment.

14. The method of claim 8 including the further step of configuring the cover of flexible solid foam material including a side wall extending up from a footing at a perimeter of the cover adjacent the skin, to a top wall spaced from the skin and overlying the inner cavity, with a door located on the top wall, the door including a hinge connecting the door to the cover in a pivotable fashion, the door including an adhesive closure perimeter adapted to abut the cover adjacent the door to sealingly close the door, the perimeter of the door formed with resealable adhesive which allows the door to be repeatedly opened and sealingly closed.

15. A transcutaneous vascular access point dressing assembly, comprising in combination:
an adhesive membrane having an adhesive undersurface adapted to contact and adhere to skin and an over surface opposite said undersurface;
a cover attached to said over surface of said adhesive membrane;
said cover substantially enclosing an inner cavity above said adhesive membrane and beneath portions of said cover; and
a selectively closable entrance into said inner cavity, for access to items stored within said inner cavity.

16. The dressing assembly of claim 15 wherein said cover is at least partially formed of a flexible material.

17. The dressing assembly of claim 16 wherein said cover is at least partially formed of a solid foam material.

18. The dressing assembly of claim 15 wherein said entrance includes a door in said cover, said inner cavity accessible through said door when said door is open.

19. The dressing assembly of claim 18 wherein said cover includes a sidewall surrounding a perimeter of said inner cavity, said side wall extending up from a footing attached to said adhesive membrane to a top wall spaced from said adhesive membrane and overlying said inner cavity, with said door located on said top wall.

20. The dressing assembly of claim 19 wherein said door is hinged to said cover on a portion of said door, such that said door can pivot about said hinge to selectively close and open access to said inner cavity.

21. The dressing assembly of claim 20 wherein said door has a perimeter with adhesive thereon, said perimeter of said door sized larger than an opening in said cover adjacent said door, with said adhesive perimeter of said door adapted to abut said cover adjacent said door to sealingly close said door, said perimeter of said door formed with a resealable adhesive which allows said door to be repeatedly opened and sealingly closed.

22. The dressing assembly of claim 15 wherein said cover has portions thereof which are spaced from said adhesive membrane;

23. The dressing assembly of claim 15 wherein said adhesive membrane includes an aperture therein.

24. The dressing assembly of claim 23 wherein said aperture in said adhesive membrane is smaller than a size of a floor of said inner cavity, said aperture in said adhesive membrane defining a portion of said floor of said inner cavity.

25. The dressing assembly of claim 15 wherein said adhesive membrane has a surface area greater than area inboard of an outer perimeter of said cover, such that said adhesive membrane extends beyond said outer perimeter of said cover laterally for at least portions of said cover.

26. The dressing assembly of claim 25 wherein said adhesive membrane is in the form of adhesive tape with said adhesive undersurface adapted to releasably seal with said over surface of said adhesive membrane to allow unrolling of said adhesive membrane off of a spiral roll of the adhesive tape.

27. The dressing assembly of claim 25 wherein said adhesive membrane is formed at least partially of hydrocolloid material.

28. The dressing assembly of claim 25 wherein said adhesive membrane is in the form of an at least partially transparent semi-permeable membrane.

29. The dressing assembly of claim 28 wherein said adhesive membrane includes thin polyurethane film.
